(19) United States
(12) Patent Application Publication
(10) Pub. No.: US 2014/0074009 A1
(43) Pub. Date: Mar. 13, 2014

(54) METHOD FOR GENERATING A SINGLET OXYGEN

(71) Applicants: Kuo-Chu HWANG, Hsinchu City (TW); Raviraj Vankayala, Hsinchu City (TW)

(72) Inventors: Kuo-Chu HWANG, Hsinchu City (TW); Raviraj Vankayala, Hsinchu City (TW)

(21) Appl. No.: 13/615,409

(22) Filed: Sep. 13, 2012

(51) Int. Cl. A61M 37/00; B82Y 5/00

(52) U.S. Cl. 604/20; 977/773; 977/906

(57) ABSTRACT

The invention provides a method for generation of a singlet oxygen comprising the following steps: irradiating with light on a metal nanoparticle at a specific wavelength; and transmitting a photon energy to sensitize a molecular oxygen to generate the singlet oxygen; wherein, the amount of the singlet oxygen is dependent on the wavelength of excitation light and aspect ratio of the metal nanoparticle.

### BACKGROUND OF THE INVENTION

1. Field of the Invention

The present invention relates to a method for generating a singlet oxygen, and more particularly to a method for generating a singlet oxygen through irradiating with light on a metal nanoparticle, or metal nanorod or a metal nanoshell.

2. Related Art

Singlet oxygen (¹O₂) is known to play an indispensible role in the photodynamic therapy (PDT) treatment of cancer, and is an important oxidant for hydroperoxidation of olefins in organic synthesis. Singlet oxygen is conventionally formed by sensitization of organic photosensitizers, such as Rose Bengal, silicon phthalocyanine, etc. These organic or organometallic dyes are, however, prone to photo-induced degradation and enzymatic degradation, which becomes problematic in PDT treatments, and reduces the efficiency of the generation of singlet oxygen. It is, therefore, important to search for photosensitizers with highly efficient singlet oxygen generation and large absorption coefficients that are photochemically more stable and less prone to enzymatic degradation.

### SUMMARY OF THE INVENTION

An objective of the present invention is to provide a method for generating a singlet oxygen.

An objective of the present invention is to provide a method can increase an amount of reactive oxygen species.

Another objective of the present invention is to provide a method to destroy DNA of a cell according to the singlet oxygen.

An embodiment of the invention provides a method for generating a singlet oxygen comprising the following steps: irradiating with light on a metal nanoparticle, or metal nanorod or a metal nanoshell at a specific wavelength; and transmitting photon energy to excite an oxygen molecule to generate the singlet oxygen; wherein, amount of the singlet oxygen is dependent on the wavelength of light and aspect ratio of the metal nanoparticle.

### BRIEF DESCRIPTION OF THE DRAWINGS

The present invention will become more fully understood from the detailed description given herein below and the accompanying drawings which are given by the way of illustration only, and thus are not limitative of the present invention.

FIG. 1A is a schematic illustration according to an embodiment of the invention.

FIG. 1B is a TEM (Transmission Electron Microscopy) image of gold nanorod.

FIG. 2A shows phosphorescence emission spectra of singlet oxygen sensitized by Au, Ag or Pt nanoparticles in D₂O.

FIG. 2B shows the extinction spectra (solid lines) of Ag, Pt, and Au NPs.

FIG. 3 is a bar chart showing that relationship between means fluorescence intensity of SOSG and concentration of M NPs.

FIG. 4A is a reaction scheme of cyclohexene with singlet oxygen.

FIG. 4B is ¹H NMR spectra of the photoinduced peroxidation product of cyclohexene in dichloromethane-acetonitrile (100 W high-pressure Hg lamp) in the presence of Ag NPs, Pt NPs, Au NPs, or in the absence of metal NPs.

FIG. 5 shows fluorescence spectra of SOSG in D₂O in the presence of Ag NPs, Pt NPs, or Au NPs or in the absence of any metal NPs.

FIG. 6 is a schematic illustration showing that a detection of ROS inside cells.

FIG. 7 is a bar chart showing that relationship between cellular uptake and concentration of lipid-coated Au NRs.

FIG. 8A is a bar chart showing that relationship between cell viability and concentration of lipid-coated Au NRs at 4° C.

FIG. 8B is a bar chart showing that relationship between cell viability and concentration of lipid-coated Au NRs at 37° C.

FIG. 9A is a bar chart showing that relationship between mean fluorescence intensity of DCF and concentration of lipid-coated Au NRs at 37° C.

FIG. 9B is a bar chart showing that relationship between mean fluorescence intensity of DCF and concentration of lipid-coated Au NRs at 4° C.

FIG. 9C is a bar chart showing that relationship between cell expressing HSP 70 and concentration of lipid-coated Au NRs at 37° C.

FIG. 10A is a picture showing that cells were fed with PBS and irradiated with 780 nm light.

FIG. 10B is a picture showing that cells were fed with PBS and irradiated with 915 nm light.

FIG. 10C is a picture showing that cells were fed with Au NRs and irradiated with 780 nm light.

FIG. 10D is a picture showing that cells were fed with Au NRs and irradiated with 915 nm light.

FIG. 10E is a bar chart showing the mean fluorescence intensity levels of caspase-3 in PBS or Au NRs by irradiating with 780 nm and 915 nm light.

FIG. 11 is a broken line graph showing the relationship between relative tumor volume and various treatments.

### DETAILED DESCRIPTION OF THE INVENTION

The present invention will be apparent from the following detailed description, which proceeds with reference to the accompanying drawings, wherein the same references relate to the same elements.

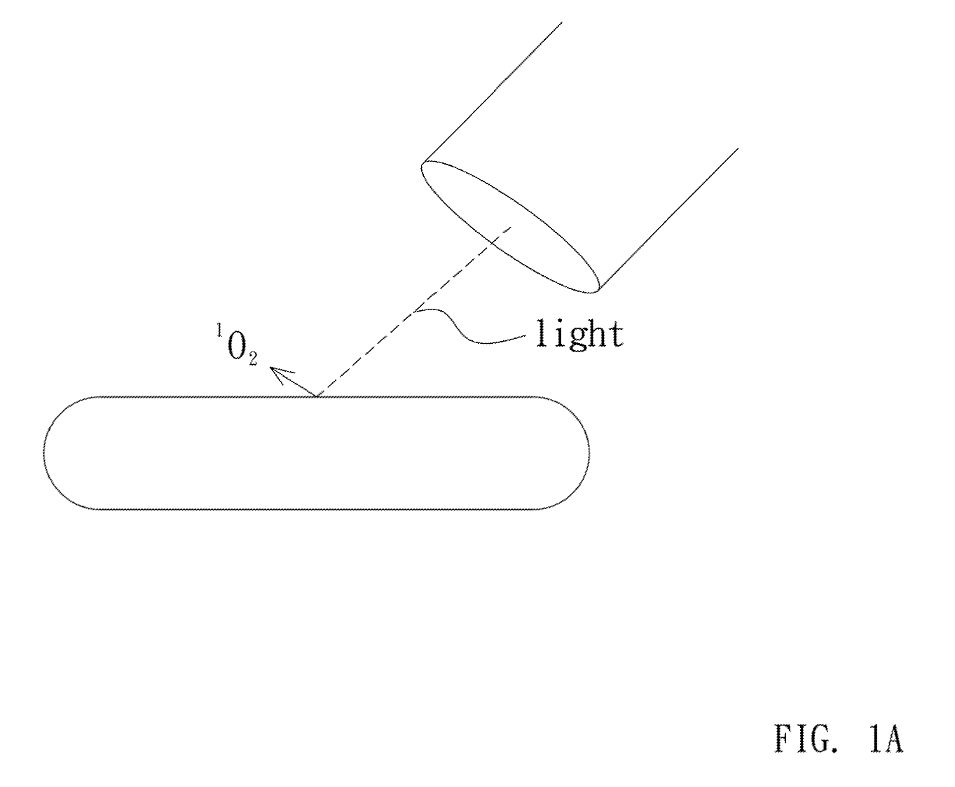

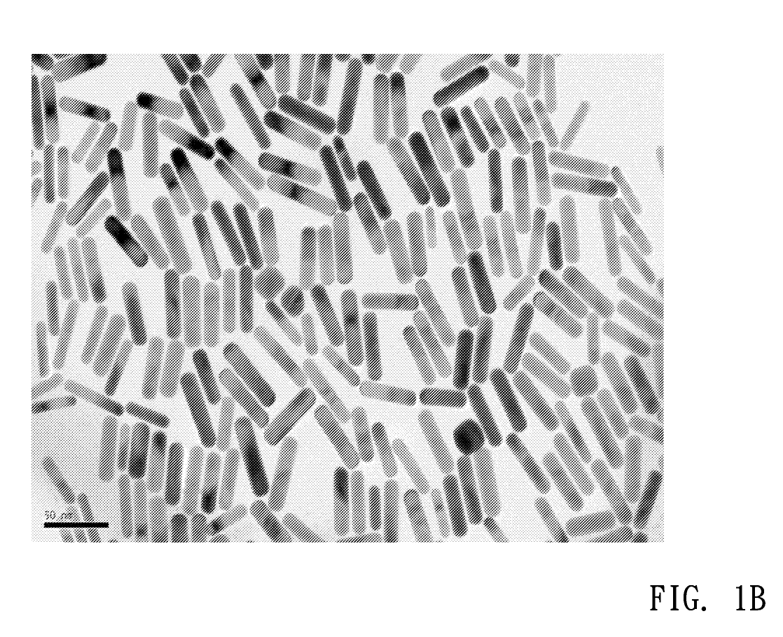

The present invention is directed to a method for generating a singlet oxygen. In one embodiment, we irradiates with light on a metal nanoparticle at a specific wavelength. However, light transmits photon energy to excite an oxygen molecule to generate the singlet oxygen. Wherein, the metal nanoparticle can be either a nanosphere, or metal nanorod, or a metal nanoshell. In another embodiment, the metal nanoparticle is a gold (Au) nanorod. Please refer to FIGS. 1A and 1B, FIG. 1A is a schematic illustration according to an embodiment of the invention; FIG. 1B is a TEM (Transmission Electron Microscopy) image of gold nanorod. In the present invention, the metal nanoparticle can also utilize silver (Ag) nanoparticle or platinum (Pt) nanoparticle.

It should be noted that, the singlet oxygen can be formed according to different wavelength corresponding to type of the metal nanoparticle. For example, when the metal nanoparticle is the gold nanorod, wavelength of light has a dimension of 550˜1300 nm. In addition to the gold nanorod, wavelength of light has a dimension of 650˜1300 nm. So that light can be near infrared light when wavelength is 650˜1300 nm.

Most importantly, the specific temperature of the method has a dimension of 0˜46° C. In other words, the singlet oxygen can be generated between 0˜46° C. Moreover, the amount of the singlet oxygen is dependent on the wavelength of excitation light and aspect ratio of the metal nanoparticle.

Herein we find that an unprecedented observation that singlet oxygen can be formed through direct sensitization by metal (Ag, Pt, or Au) nanoparticles without the presence of any organic photosensitizers. Unambiguous experimental evidence includes direct observation of singlet oxygen emission at roughly 1268 nm, hydroperoxidation of cyclohexene, green fluorescence from a selective singlet oxygen fluorescent sensor, namely, Singlet Oxygen Sensor Green (SOSG, Molecular Probe), and quenching of singlet oxygen phosphorescence by sodium azide.

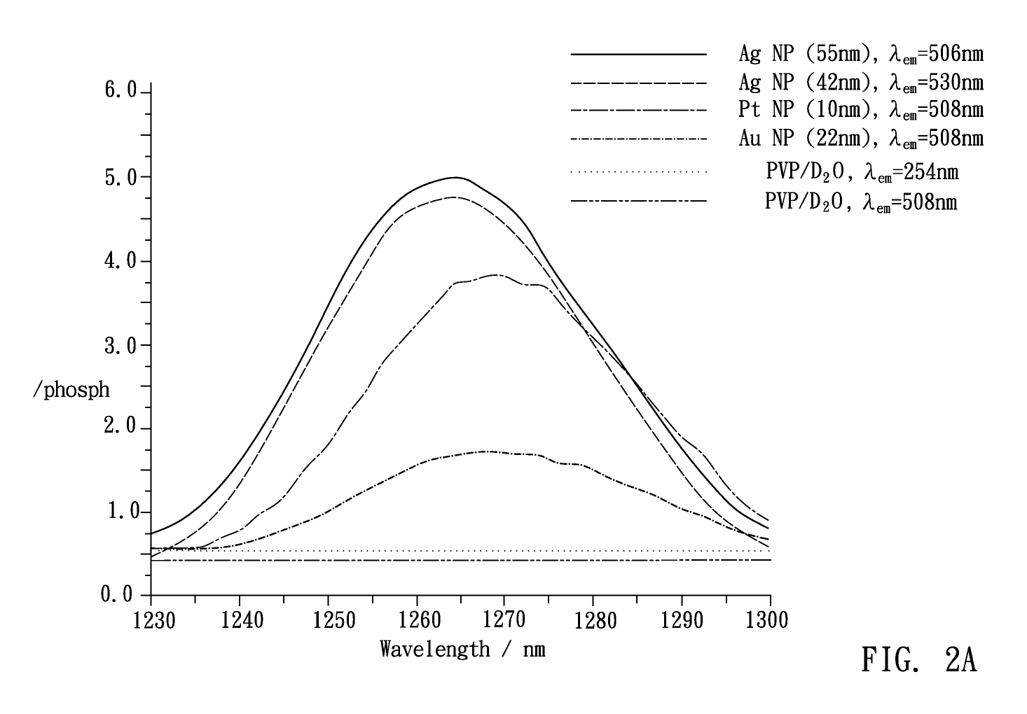

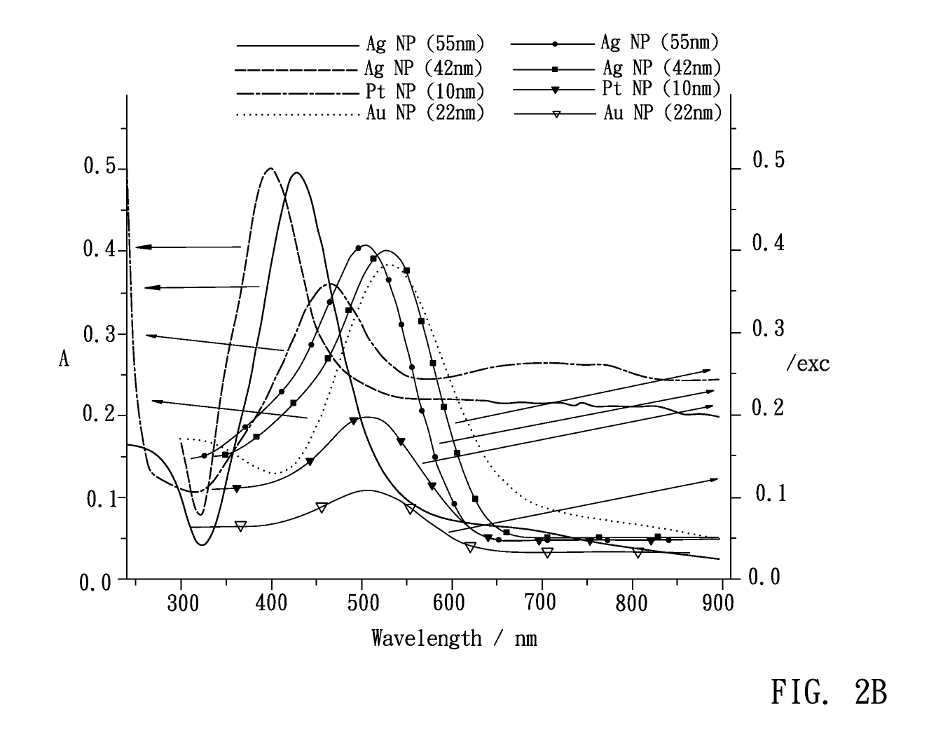

In the embodiment of the invention, emission wavelength of the singlet oxygen has a dimension of 1260˜1280 nm. Please refer to FIG. 2A and 2B. FIG. 2A shows phosphorescence emission spectra of singlet oxygen sensitized by Au, Ag or Pt nanoparticles in D₂O, and a control experiment with PVP in D₂O in the absence of metal nanoparticles (purple line, λₑₓ=254 nm and blue line, λₑₓ=508 nm). FIG. 2B shows the extinction spectra (solid lines) of Ag, Pt, and Au NPs, and the excitation spectra (dashed lines) of singlet oxygen phosphorescence at 1270 nm in the presence of Ag, Pt, and Au NPs, respectively. A longpass filter of 850 nm was put between the sample and the detector for all experiments (unless otherwise mentioned) to filter away any stray light and the second harmonic of the excitation light with wavelengths shorter than 850 nm. The excitation light profiles of 508 nm are also shown (the gray blue line was recorded without the LP850 filter).

As shown in FIG. 2A and 2B, photo-excitation of M NPs (metal nanoparticles, M NPs) at the surface plasmon resonance absorption bands of Ag (diameter d=55, 42 nm), Pt (10 nm), and Au (22 nm) in D₂O results in characteristic singlet oxygen emission at 1264 and 1268 nm, respectively. Control experiments show that in the absence of metal nanoparticles, no singlet oxygen was formed (pink and purple lines in FIG. 2A). The control experiments clearly rule out the possibility of the singlet oxygen emission signal being from stray light, scattered light, or an O₂-water charge-transfer complex. Thus photoexcitation of Ag, Pt, and Au NPs in H₂O can result in the formation of singlet oxygen. Replacement of PVP by hexadecylcetyltrimethyl ammonium bromide (CTAB) or trisodium citrate can also lead to the formation of singlet oxygen. Sodium azide is known to be a very efficient electron-transfer quencher of singlet oxygen. Control experiments also show that the singlet oxygen phosphorescence intensity becomes lower at higher concentrations of sodium azide.

All four M NPs have one major localized surface plasmon resonance (LSPR) band around 398˜530 nm (see solid lines in FIG. 2B). The maximum of the excitation spectrum (see dashed lines in FIG. 2B) shifts from 504 to 529 nm when the size of the Ag NPs decreases from 55 to 42 nm. The excitation spectra show that excitation at wavelengths longer than 660 nm leads to negligible amount of singlet oxygen formation for all metal nanoparticles. Comparison of the LSPR of metal nanoparticles and the excitation spectra of singlet oxygen emission (see FIG. 2B) shows that the low-energy surface states of the metal nanoparticles can transfer energy to molecular oxygen with high efficiency and sensitize the formation of singlet oxygen, whereas the high energy surface states of M NPs transfer LSPR energy to molecular oxygen with low efficiencies. The mismatch between the excitation spectra and the absorption spectra indicates that metal nanoparticles, similar to azulene and their derivatives, do not follow Kashas rule, and their plasmonically excited state behavior is strongly dependent on the excitation wavelengths. Such a result is attributed to the fact that the absorption/extinction spectrum of a metal nanoparticle is composed of many non-conjugated and localized surface plasmon resonances of different crystalline facets, and the LSPR of each metal nanoparticle behaves independently.

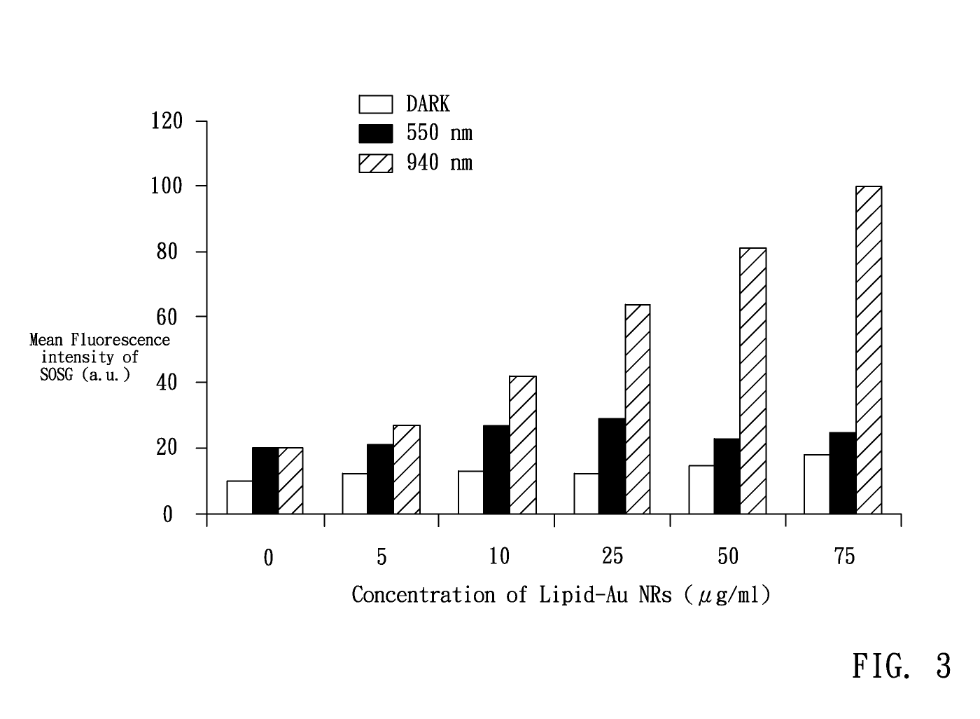

Please refer to FIG. 3. FIG. 3 is a bar chart showing that relationship between mean fluorescence intensity of SOSG and concentration of M NPs. In this embodiment, the M NPs utilizes gold nanorods (Au NRs) and the temperature is 37° C. By irradiating at different wavelengths of light (dark, 550 nm visible light, and 940 nm near infrared light), the bars of 940 nm light increase with concentration of M NPs. We can understand that the enhancement of SOSG-singlet oxygen reaction products strongly supports that the key component of ROS is singlet oxygen.

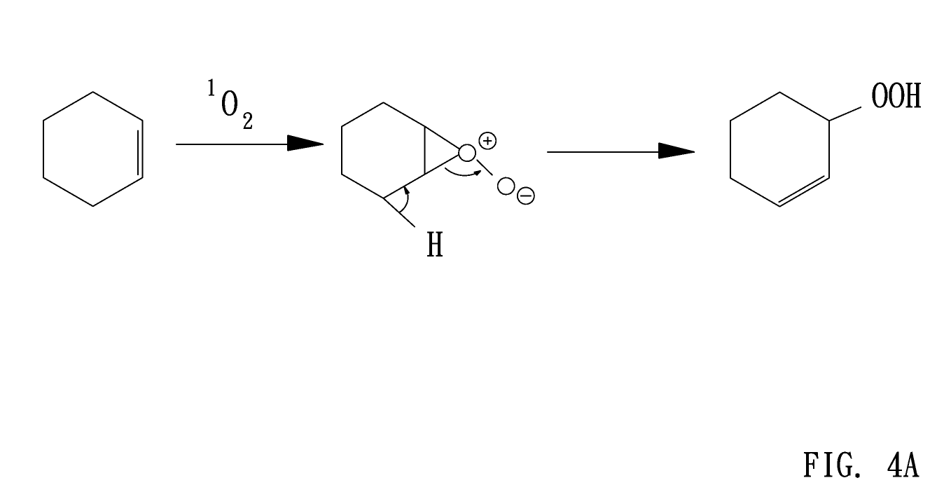

As shown in FIG. 4A and 4B, FIG. 4A is a reaction scheme of cyclohexene with singlet oxygen. FIG. 4B is ¹HNMR spectra of the photoinduced peroxidation product of cyclohexene in dichloromethane-acetonitrile (100 W high-pressure Hg lamp) in the presence of Ag NPs, Pt NPs, Au NPs, or in the absence of metal NPs. The signals of the peroxidation product, 2-hydroperoxyl cyclohexene, are assigned. In the case of Au NPs, a small amount of D₂O was added to help disperse the nanoparticles; this led D-H exchange of the R—OOH proton.

Photoirradiation of cyclohexene in dichloromethane-acetonitrile in the presence of metal nanoparticles, such as Ag NPs (d=55 nm), Au NPs (≈22 nm), and Pt NPs (≈10 nm), results in the formation of 2-hydroperoxyl cyclohexene (see FIG. 4A). The hydroperoxidation of cyclohexene to form 2-hydroperoxyl cyclohexene is known to occur in the presence of singlet oxygen. In the ¹H NMR spectrum the observation of signals for an allylic proton at δ=4.49 ppm, two vinyl protons (δ=6.0 and 5.74 ppm, multiplet), and a peroxide protons at δ=8.5 ppm indicate the formation of hydroperoxyl cyclohexene. In the case of Au NPs, the peroxide signal at δ=8.5 ppm was absent, because of H-D exchange with the D₂O added to the solution to help disperse the nanoparticles. Formation of hydroperoxyl cyclohexene unambiguously supports the formation of singlet oxygen upon photoirradiation of M NPs. From the integration of the ¹H NMR spectra of the hydroperoxyl cyclohexene product (relative to an internal standard, 1,4-dicyanobenzene), it is clear that Ag NPs are more efficient than Pt and Au NPs at sensitizing the formation of singlet oxygen.

To further confirm that M NPs can indeed sensitize the formation of singlet oxygen upon photoirradiation, we used commercial singlet oxygen sensor, SOSG to trap singlet oxygen. The chemical structure of SOSG was not disclosed, but it is believed to be an anthracene-fluorescein derivative. SOSG has been demonstrated to have a very good selectivity towards singlet oxygen, and does not show any noticeable response towards hydroxyl radicals or superoxide. Upon reaction with singlet oxygen to form endoperoxide, SOSG shows green fluorescence with an emission maximum at 525 nm.

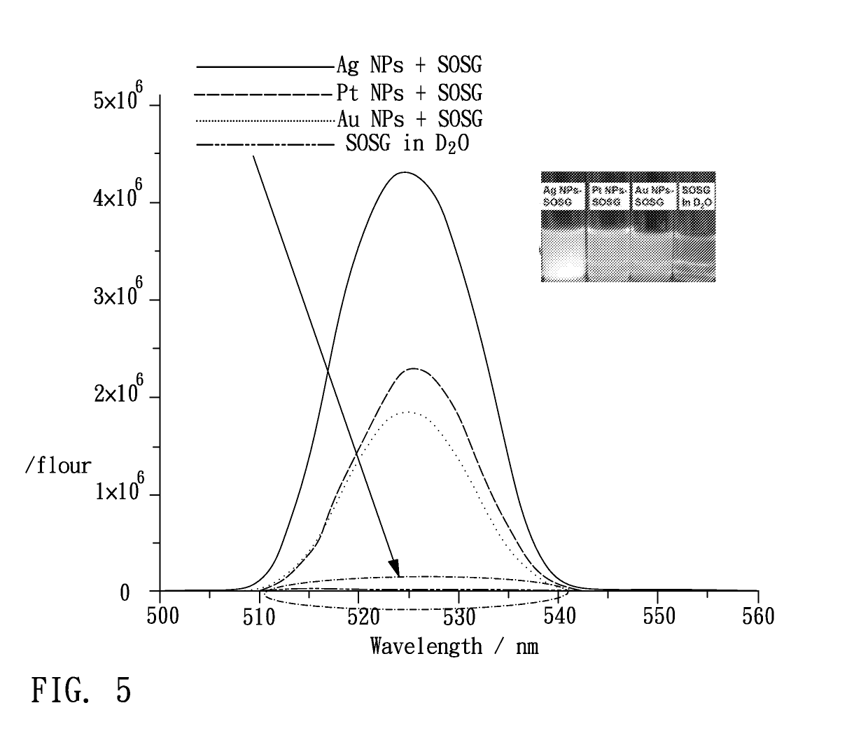

As shown in FIG. 5, FIG. 5 shows fluorescence spectra of SOSG in D₂O in the presence of Ag NPs, Pt NPs, or Au NPs or in the absence of any metal NPs. The solutions were irradiated with a 100 W high-pressure Hg lamp for 2 min; excitation wavelength 382 nm. The inset shows green fluorescence emission from SOSG in four different D₂O solutions in the presence and absence of metal nanoparticles. Photoirradiation of metal NPs, including Ag, Pt, and Au NPs in D₂O in the presence of SOSG for 2 min results in the formation of strongly fluorescent SOSG (λₑₘ=525 nm). The results clearly indicate that photoirradiation of metal nanoparticles indeed can result in the formation of singlet oxygen. In a control experiment, SOSG alone was irradiated in D₂O in the absence of metal nanoparticles; green fluorescence was observed, but with far weaker intensity (the black circle in FIG. 5). The inset pictures in FIG. 3 show the relative brightness of SOSG fluorescence in the presence of different M NPs. Our data (FIG. 5) show that the amount of singlet oxygen formed through the sensitization of SOSG is negligible compared to the amounts of singlet oxygen formed through sensitization by M NPs.

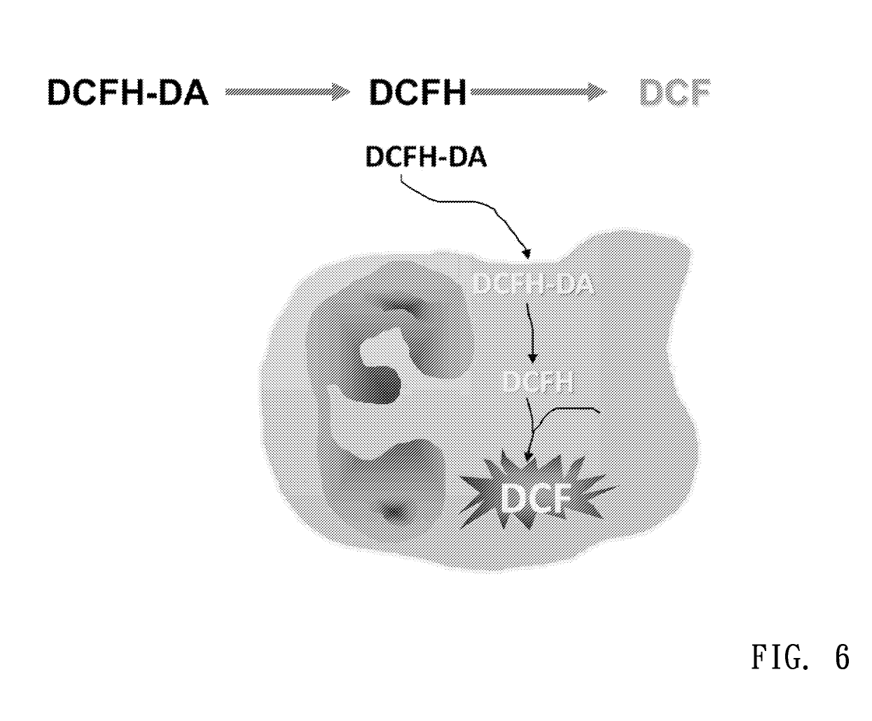

In one embodiment of the invention, the method comprising that singlet oxygen can increases an amount of reactive oxygen species (ROS). Please refer to FIG. 6, FIG. 6 is a schematic illustration showing that a detection of ROS inside cells. The intracellular ROS generation of cells can be investigated using DCFH-DA (2′,7′-dichlorfluorescein diacetate) as a well-established compound to detect and quantify intracellular H₂O₂. The conversion of non-fluorescent DCFH-DA to the highly fluoresecent compound DCF (2′,7′-dichlorfluorescein) happens in several steps. First, DCFH-DA is transported across the cell membrane and deacetylated by esterases to form the non-fluorescent DCFH. This compound is trapped inside of the cells. Next, DCFH is converted to DCF through the action of peroxidase rated by the presence of peroxidase. When DCF is photochemically excited by a specific wavelength of light, it will emit a green fluorescence.

In the embodiment, there are several steps for ROS detection. Step 1: Load cells of 2×10⁵ cells/mL, and incubate for 24 hours to let it stick on the plate. Step 2: Wash the cell with PBS (phosphate buffer solution) for one time and replace the medium with reduced serum medium and then add lipid-coated Au NRs, and let it uptake for 4 hours. Step 3: Simultaneously maintain the cells under dark and photoirradiation condition 940 nm and 550 nm for 40 and 120 minutes. Step 4: Immediately after irradiation, feed the cells with DCFH-DA and incubate at 37° C. for 30 minutes, and then the cells were prepared for flow cytometry measurements.

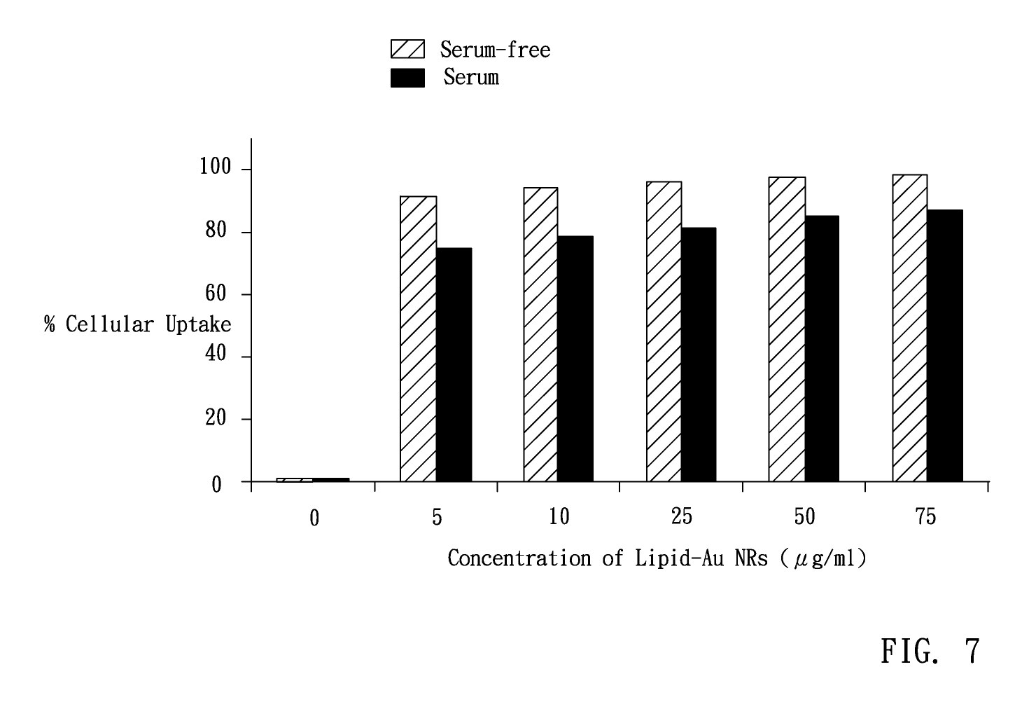

Please also refer to FIG. 7, FIG. 7 is a bar chart showing that relationship between cellular uptake and concentration of lipid-coated Au NRs. Wherein, red bar shows that the cells will absorb lipid-coated Au NRs when there is no serum (red bars shows serum-free). Therefore, we can know that lipid-coated Au NRs is likely to be uptaken by the cells, and the cellular uptake is proportional to concentration of lipid-coated Au NRs.

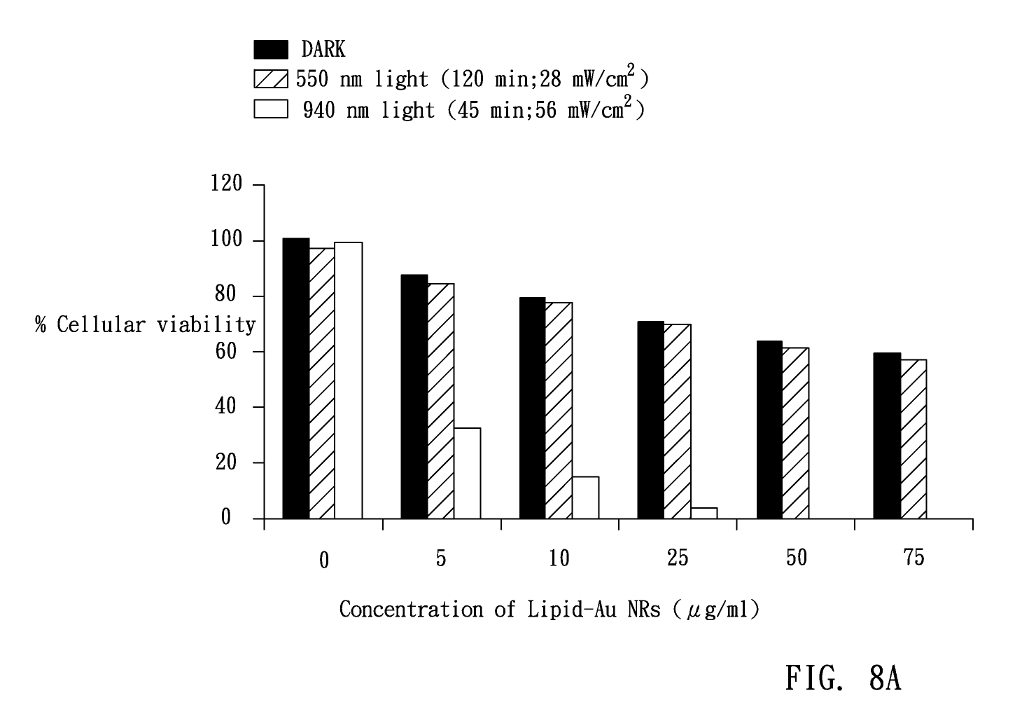

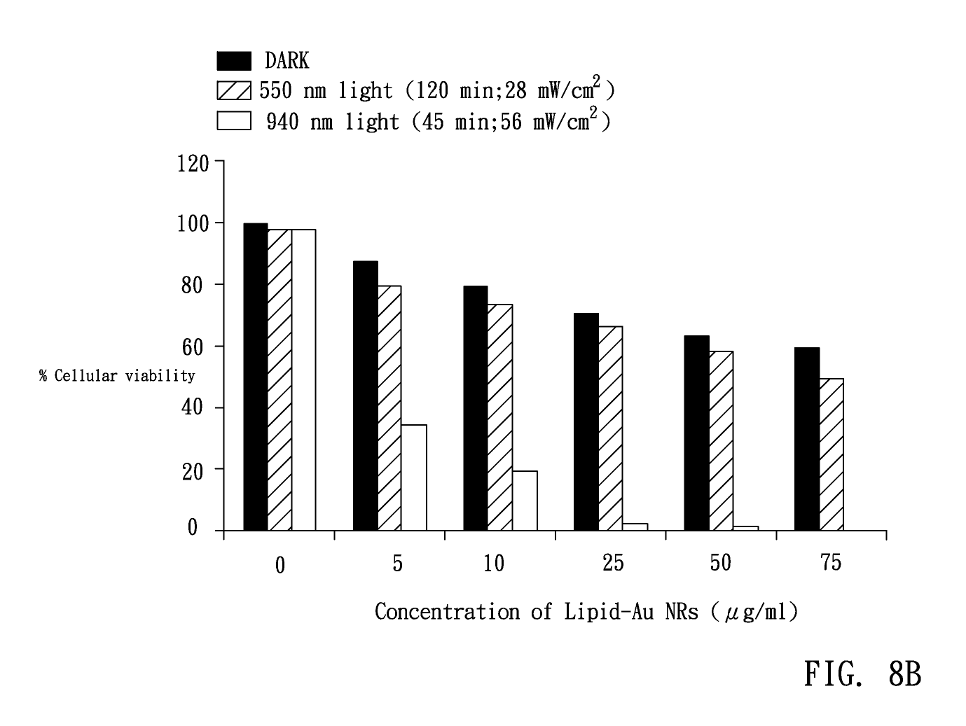

Please also refer to FIG. 8A and 8B. FIG. 8A is a bar chart showing that relationship between cell viability and concentration of lipid-coated Au NRs under 4° C., FIG. 8B is a bar chart showing that relationship between cell viability and concentration of lipid-coated Au NRs at 37° C. In the invention, when the ROS is increasing, DNA (deoxyribonucleic acid) of a cell will be destroyed through ROS. In other words, accumulation of ROS causes the breakage of DNA, protein and lipid membranes membranous lipids, resulting in irreversible cellular damages. It shows that the cells will be rapidly destructed when irradiating with light (wavelength is 940 nm) on Au NRs. It should be noted that, 4° C. and 37° C. are not in the operating range of photothermal therapy. Therefore, the cells are destroyed according to singlet oxygen formed via sensitization of molecular oxygen by plasmonically excited Au NRs.

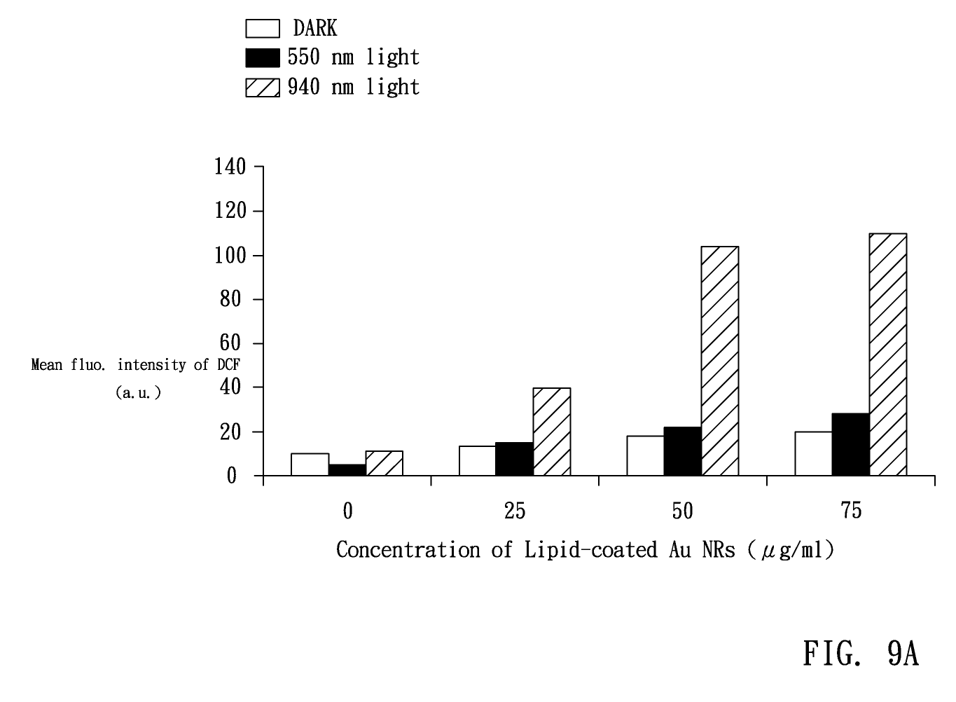

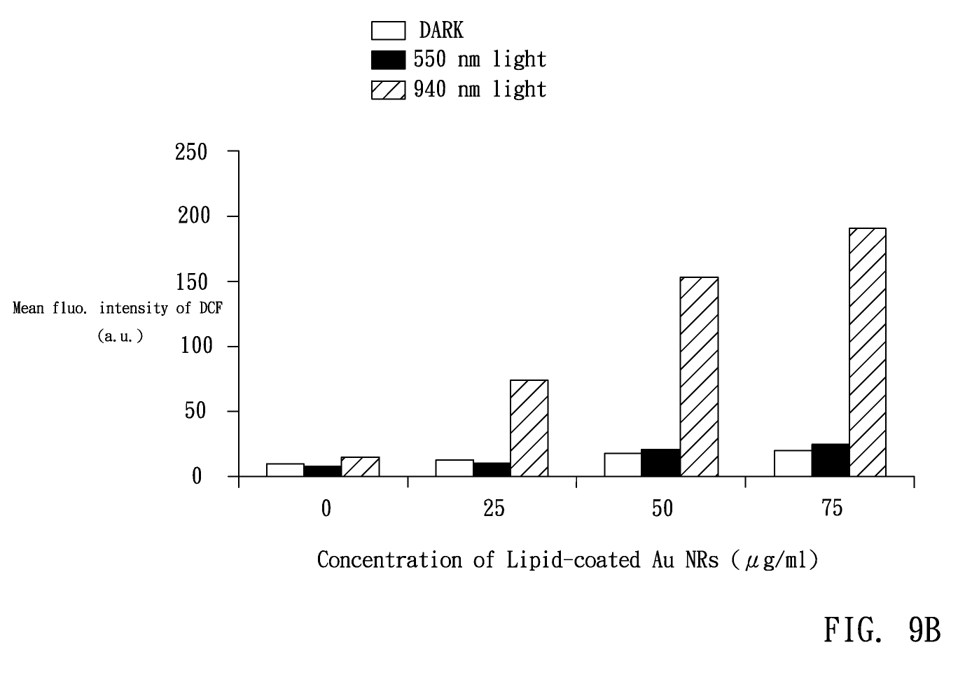

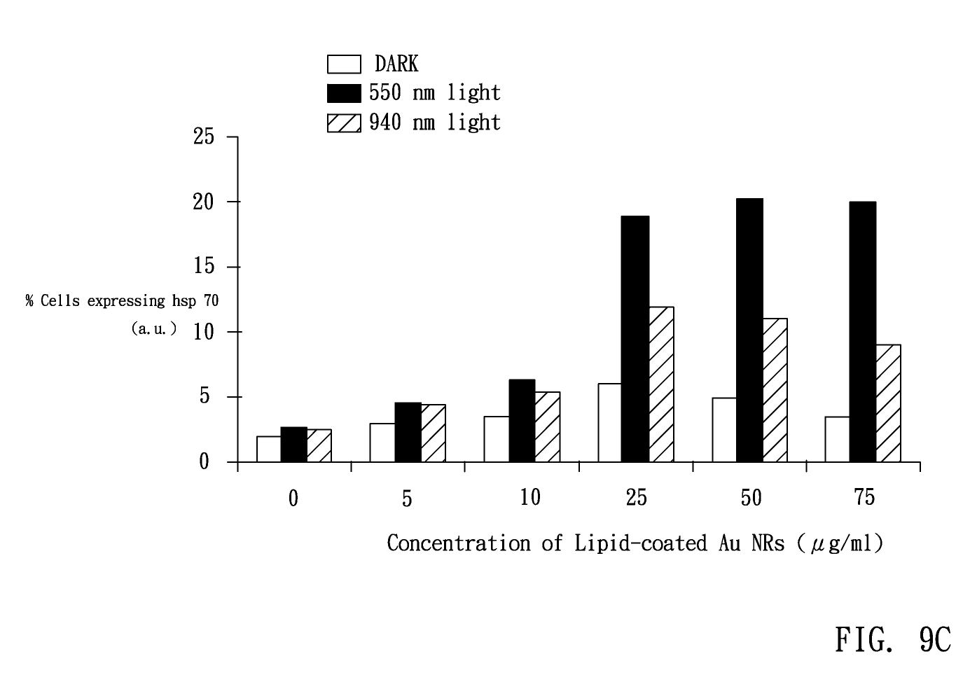

In order to prove that ROS is increasing, we do the following experiment. Please also refer to FIG. 9A and 9B. FIG. 9A is a bar chart showing that relationship between mean fluorescence intensity of DCF and concentration of lipid-coated Au NRs at 37° C. FIG. 9B is a bar chart showing that relationship between mean fluorescence intensity of DCF and concentration of lipid-coated Au NRs under 4° C. When irradiating with 940 nm light, the mean fluorescence intensity of DCF (see bars of 940 nm light) are greater than dark or 540 nm light. Therefore, Au NRs can sensitize formation of singlet oxygen by irradiating with near infrared light. In one embodiment of the invention, position of the gold nanorod through fluorescence can be positioned according to fluorescence emitted from gold nanorod itself.

It should be noted that, the 70 kilodalton heat shock proteins (HSP 70) are a family of ubiquitously expressed heat shock proteins. Proteins with similar structure exist in virtually all living organisms. The HSP70 is an important part of the cell's machinery for protein folding, and help to protect cells from stress. In the invention, we also do HSP70 protein expression analysis at 37° C. Please refer to FIG. 9C. FIG. 9C is a bar chart showing that relationship between cell expressing HSP70 and concentration of lipid-coated Au NRs under 37° C. It can understand that by irradiating with 550 nm light, cell expressing HSP70 is indeed formed with an amount more than those by irradiation using 940 nm light or in dark. It can be explained that the cells indeed been thermally destructed when irradiating with 550 nm light.

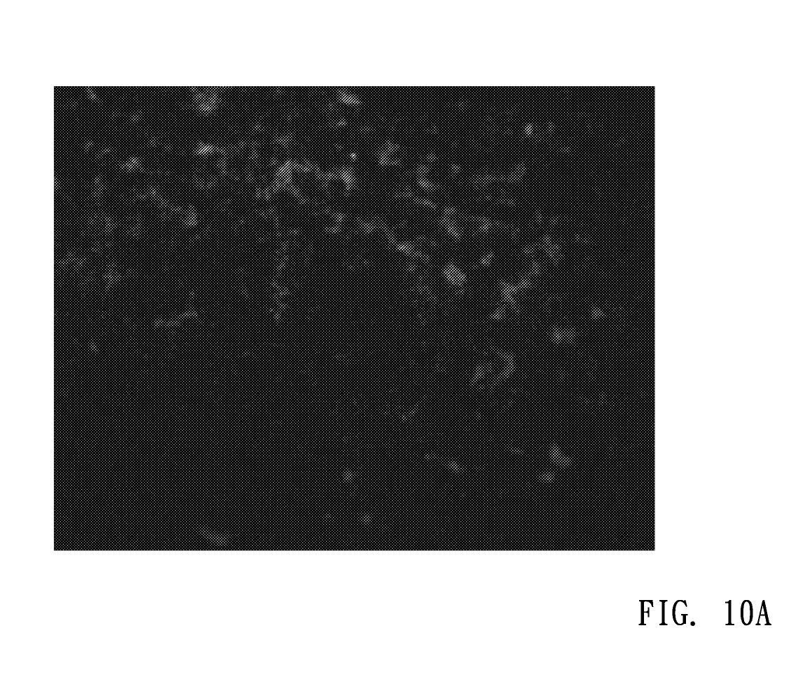

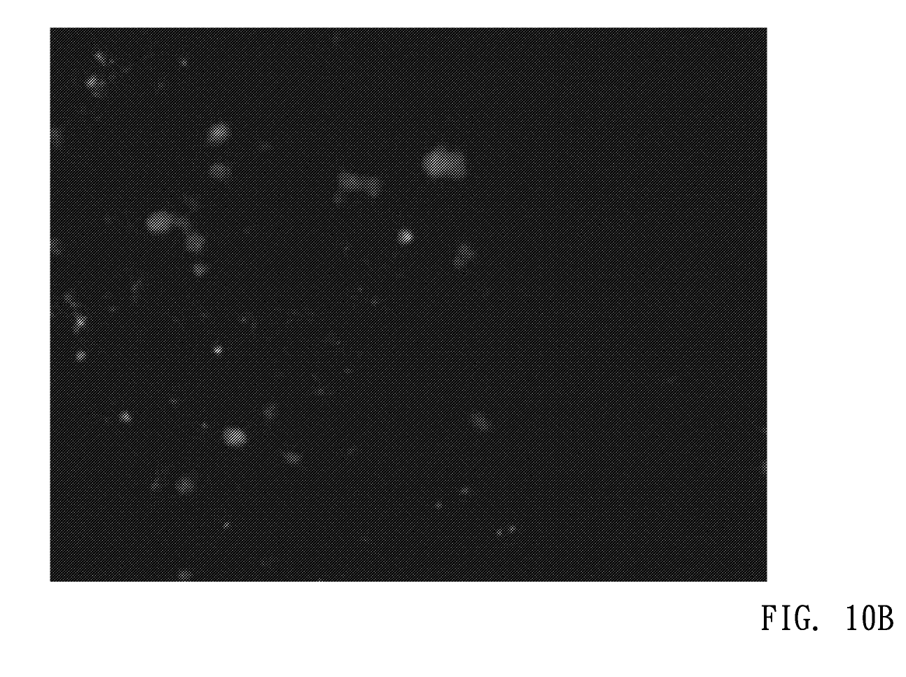

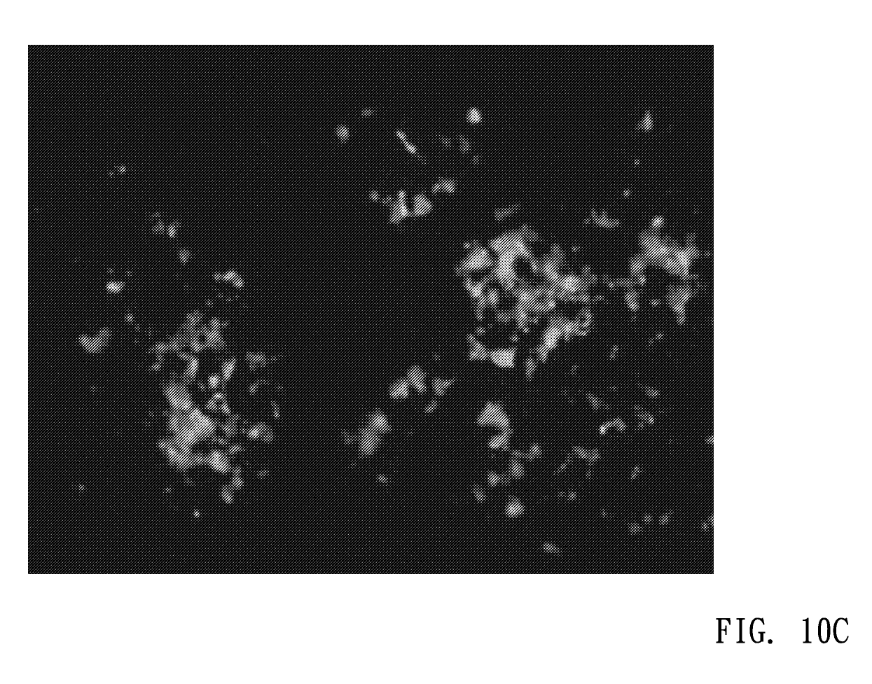

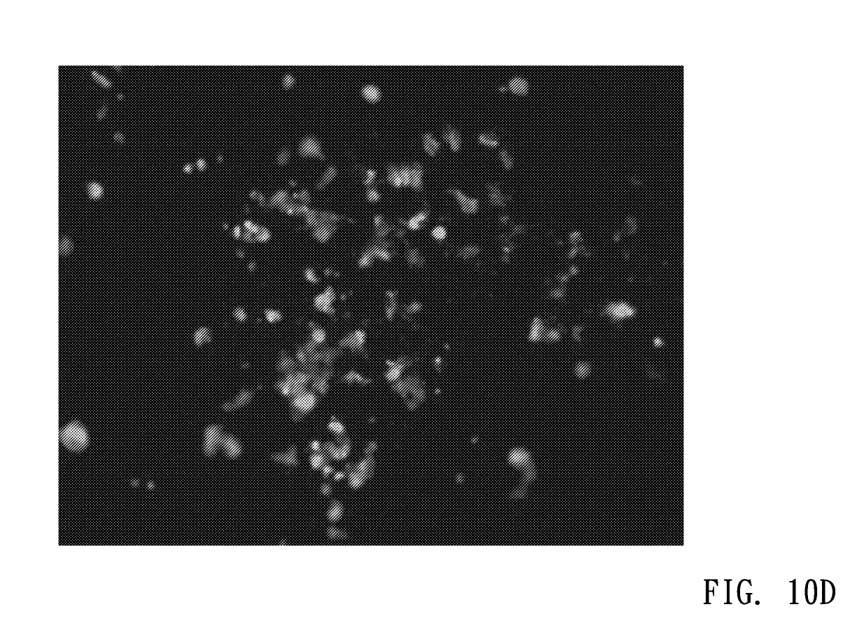

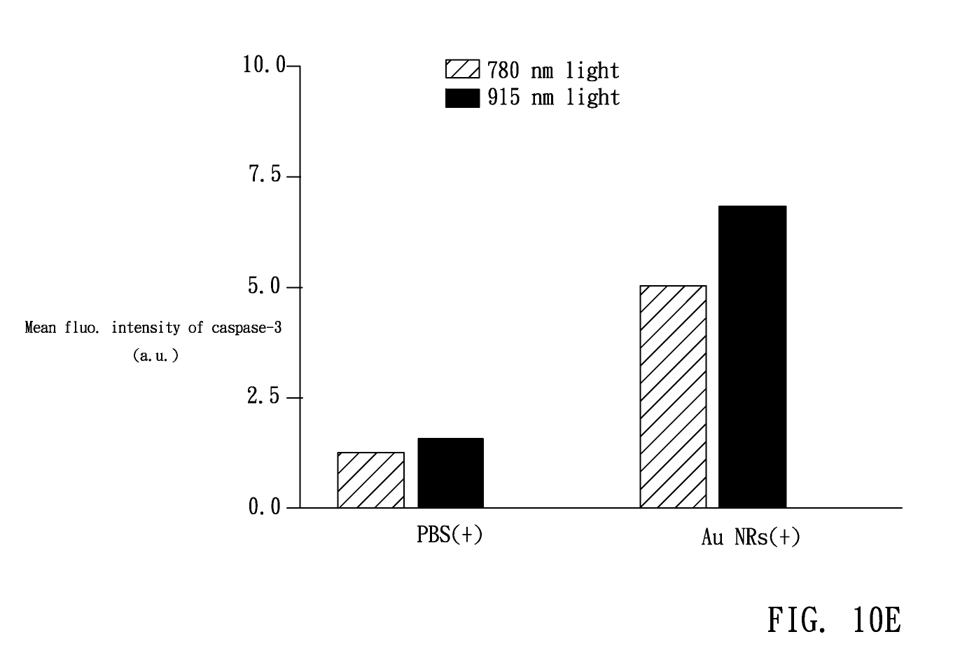

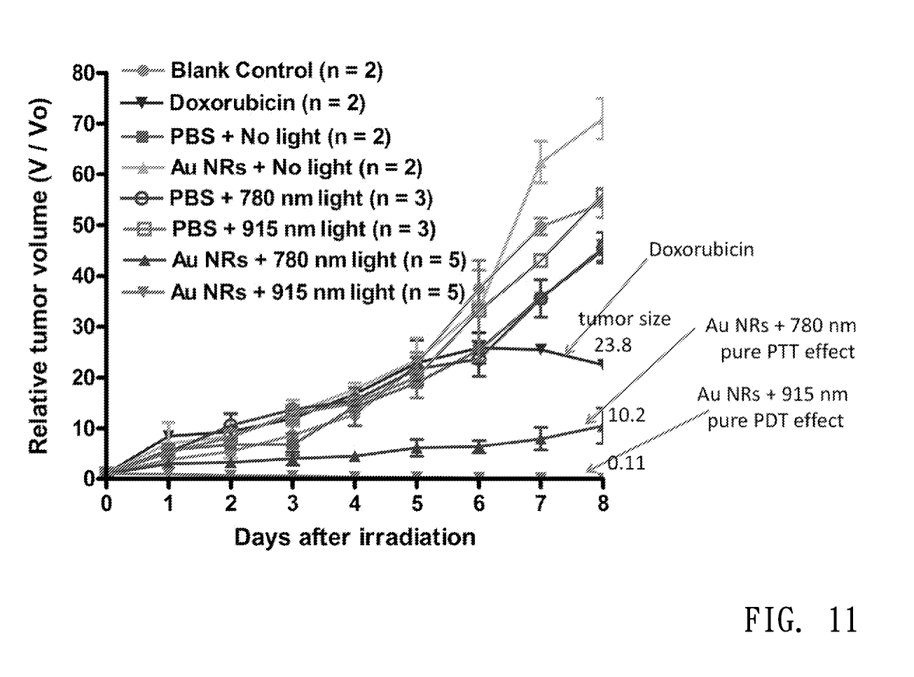

Furthermore, singlet oxygen induce caspase-3 activation in prior art. We also do caspase-3 staining for apoptosis detection in this embodiment. Please refer to FIG. 10A˜10E. FIG. 10A is a picture showing that cells were fed with PBS and irradiated with 780 nm light; FIG. 10B is a picture showing that cells were fed with PBS and irradiated with 915 nm light; FIG. 10C is a picture showing that cells were fed with Au NRs and irradiated with 780 nm light; FIG. 10D is a picture showing that cells were fed with Au NRs and irradiated with 915 nm light; and FIG. 10E is a bars chart showing that mean fluorescence intensity of caspase-3 are in PBS or Au NRs by irradiating with 780 nm light and 915 nm light. Wherein, the amount of Caspase-3 is proportional to the amount of singlet oxygen. Therefore, the amount of Caspase-3 is substantially increased when irradiating with 780 nm or 915 nm light. Through the detection, singlet oxygen induces Caspase-3 activities directly.

Above mentioned, the cells also can be cancer cells. In other words, the present invention should be applied to any cells. Therefore, in one embodiment of the invention, we make B16F0 melanoma model was injected into the subcutaneous region of each mouse. Please refer to FIG. 11, FIG. 11 is a broken line graphs showing that relationship between relative tumor volume and various treatments. Wherein, in FIG. 11, n represents the number of mice.

It should be noted that, in this experiment of the embodiment, we utilize pure photothermal therapy, Doxorubicin and pure photodynamic therapy on B16F0 melanoma model. Wherein, pure photothermal therapy is achieved by irradiation of Au NRs using 780 nm light to produce a local temperature in tumor to be over 46° C.; and pure photodynamic therapy is achieved by irradiation of Au NRs using 915 nm light to generate singlet oxygen at a temperature between 0˜46° C. As shown in FIG. 11, pure photodynamic therapy increases the cancer cellular death rate after eight days of treatment. The relative tumor volume is controlled under 0.11. It can be understood that the tumor size is actually decrease under photodynamic therapy (915 nm light on Au NRs between 0˜46° C.). In other treatments, for example: Au NRs without irradiating with light, or PBS and irradiation with light, or PBS without light irradiation, the results are not as good as that in the case of pure photodynamic therapy.

When under photothermal therapy effect, it destroys cancer cells through a high temperature (high temperature is greater than 46° C.). However, local temperature has to reach at least 46° C. Such a local temperature inside a cancer cell is different from the temperature in the outside environment or biological body. Therefore, one can irradiate mice at a body temperature of either 37 or 20° C. Upon being irradiated by 780 nm light, Au NRs may completely convert photon energy to thermal energy, which will cause increase in the local temperature inside cancer cells. If the mice body temperature is 20° C., it will become much more difficult to raise the local temperature inside a tumor cell to be beyond 46° C., as compared to the case where mice body temperature is 37° C. Body temperature (or the global environment temperature) is different from the local temperature inside a cancer cell. Therefore, if one lowers down the mice body temperature, the photothermal therapy effect will be strongly suppressed, whereas the photodynamic therapy (or singlet oxygen effect) will not be affected by the environment temperature.

In summary, the method generates singlet oxygen by irradiation with a specific wavelength of light on a metal nanoparticle at a specific temperature. However, singlet oxygen can increase the amount of ROS to destroy the DNA of cancer cells. Through the aforementioned experiments, cells are destroyed by single oxygen. Moreover, in the invention, rate of cell death is greater than that in the case of pure photothermal therapy.

While the present invention has been described by the way of examples and in terms of preferred embodiments, it is to be understood that the present invention is not limited thereto. To the contrary, it is intended to cover various modifications. Therefore, the scope of the appended claims should be accorded the broadest interpretation so as to encompass all such modifications.

1. A method for generation of a singlet oxygen comprising:
irradiating a metal nanoparticle with light at a specific wavelength; and
sensitizing molecular oxygen to generate singlet oxygen upon light irradiation;
wherein the amount of singlet oxygen generated is dependent on the wavelength of the incident light and the aspect ratio of metal nanoparticles.

2. The method according to claim 1, wherein the incident light is near infrared light.

3. The method according to claim 1, wherein the specific temperature has a dimension of 0-46° C.

4. The method according to claim 2, wherein the wavelength of near infrared light has a dimension of 650-1300 nm.

5. (canceled)

6. The method according to claim 1, further comprising:
increases increasing an amount of reactive oxygen species (ROS) by adjusting the wavelength of the incident light or the aspect ratio of metal nanoparticles; and
destroying a cell's DNA through the ROS.

7. The method according to claim 2, wherein the metal nanoparticle is a gold nanorod, further comprising:
generating fluorescence by irradiating with near infrared light on the gold nanorod; and
tracking the position of the gold nanorod through the fluorescence.

8. The method according to claim 7, wherein the cell is a cancer cell.

9. The method according to claim 1, wherein the metal nanoparticle is a metal nanorod or a metal nanoshell.

10. The method according to claim 9, wherein wavelength of light has a dimension of 550-1300 nm when the metal nanoparticle is a gold nanorod.
